(19) United States
(12) Patent Application Publication
(10) Pub. No.: US 2026/0168972 A1
(43) Pub. Date: Jun. 18, 2026

(54) CALIBRATION OF MERCURY DETECTION SYSTEMS

(71) Applicants: Seth Neeley Lyman, Logan, UT (US); Trevor Legrand O'Neil, Vernal, UT (US)

(72) Inventors: Seth Neeley Lyman, Logan, UT (US); Trevor Legrand O'Neil, Vernal, UT (US)

(73) Assignee: Utah State University, Logan, UT (US)

(21) Appl. No.: 19/362,068

(22) Filed: Oct. 17, 2025

Related U.S. Application Data
(60) Provisional application No. 63/734,648, filed on Dec. 16, 2024.

(51) Int. Cl. G01N 33/00; G01N 1/38; G01N 1/40

(52) U.S. Cl. CPC G01N 33/0006; G01N 1/38; G01N 1/4055; G01N 33/0016; G01N 33/0018; G01N 33/0021; G01N 33/0045; G01N 33/0073; G01N 2001/4066

(57) ABSTRACT

The present disclosure includes a method for calibrating a mercury detection system. The method introducing a gas into a chamber. The gas passes a container within the chamber to form a mixture of mercury from the container and gas. The method includes carrying the mixture of gas and mercury out of the chamber via an input line. The method includes pulling a portion of the mixture out of the input line. The method includes measuring a flow rate of a remaining portion of the mixture in the input line and introducing the remaining portion into an analyzer via an outlet line.

### CROSS-REFERENCES TO RELATED APPLICATIONS

This application claims the benefit of U.S. Provisional Patent Application No. 63/734,648 entitled “CALIBRATION OF MERCURY DETECTION SYSTEMS” and filed on Dec. 16, 2024, for Seth Neely Lyman et al., which is incorporated herein by reference.

### GOVERNMENT RIGHTS

This invention was made with government support under 2044537 awarded by the National Science Foundation. The government has certain rights in the invention.

### FIELD

This invention relates to mercury detection and more particularly relates to calibration of mercury detection systems.

### BACKGROUND

Mercury detection systems are critical components in various industrial, environmental, and public health applications, including emissions monitoring from power plants, environmental sampling, and safety procedures. The accurate measurement of mercury, often present at trace levels, is paramount. Periodic and reliable calibration for these detection systems helps to ensure the integrity and accuracy of the data they produce. Mercury calibration systems produce precise, known concentrations of mercury in a fluid to calibrate a mercury detector.

### SUMMARY

The present disclosure includes a method for calibrating a mercury detection system. The method includes introducing a gas into a chamber. The gas passes a container within the chamber to form a mixture of mercury from the container and gas. The method includes carrying the mixture of gas and mercury out of the chamber via an input line. The method includes pulling a portion of the mixture out of the input line. The method includes measuring a flow rate of a remaining portion of the mixture in the input line and introducing the remaining portion into an analyzer via an outlet line.

The present disclosure includes a system for calibrating a mercury detection system. The system includes a chamber, a container positioned within the chamber, and an input line connected to the chamber. The system includes a controller configured to introduce a gas into the chamber such that the gas passes the container to form a mixture of mercury from the container and gas. The controller is also configured to push the mixture of gas and mercury out of the chamber via the input line. The system includes a pump configured to pull a portion of the mixture out of the input line. The system includes a flow meter configured to measure a flow rate of a remaining portion of the mixture in the input line. The system includes an outlet line configured to introduce the remaining portion of the mixture into an analyzer.

### BRIEF DESCRIPTION OF THE DRAWINGS

In order that the advantages of the invention will be readily understood, a more particular description of the invention briefly described above will be rendered by reference to specific examples that are illustrated in the appended drawings. Understanding that these drawings depict only typical examples of the invention and are not therefore to be considered to be limiting of its scope, the invention will be described and explained with additional specificity and detail through the use of the accompanying drawings, in which:

FIG. 1 is a schematic diagram illustrating a system for calibrating a mercury detection system, according to various examples;

FIG. 2 is a schematic diagram illustrating a system for calibrating a mercury detection system using additional air flow, according to various examples;

FIG. 3 is a schematic flow chart diagram illustrating a method for calibrating a mercury detection system, according to various examples; and

FIG. 4 is a schematic flow chart diagram illustrating a method for calibrating a mercury detection system using additional air flow, according to various examples.

### DETAILED DESCRIPTION

Reference throughout this specification to “one example,” “an example,” or similar language means that a particular feature, structure, or characteristic described in connection with the example is included in at least one example. Thus, appearances of the phrases “in one example,” “in an example,” and similar language throughout this specification may, but do not necessarily, all refer to the same example, but mean “one or more but not all examples” unless expressly specified otherwise. The terms “including,” “comprising,” “having,” and variations thereof mean “including but not limited to” unless expressly specified otherwise. An enumerated listing of items does not imply that any or all of the items are mutually exclusive and/or mutually inclusive, unless expressly specified otherwise. The terms “a,” “an,” and “the” also refer to “one or more” unless expressly specified otherwise.

Furthermore, the described features, structures, or characteristics of the invention may be combined in any suitable manner in one or more examples. In the following description, numerous specific details are provided, such as examples of programming, software modules, user selections, network transactions, database queries, database structures, hardware modules, hardware circuits, hardware chips, etc., to provide a thorough understanding of examples of the invention. One skilled in the relevant art will recognize, however, that the invention may be practiced without one or more of the specific details, or with other methods, components, materials, and so forth. In other instances, well-known structures, materials, or operations are not shown or described in detail to avoid obscuring aspects of the invention.

The schematic flow chart diagrams included herein are generally set forth as logical flow chart diagrams. As such, the depicted order and labeled steps are indicative of one example of the presented method. Other steps and methods may be conceived that are equivalent in function, logic, or effect to one or more steps, or portions thereof, of the illustrated method. Additionally, the format and symbols employed are provided to explain the logical steps of the method and are understood not to limit the scope of the method. Although various arrow types and line types may be employed in the flow chart diagrams, they are understood not to limit the scope of the corresponding method. Indeed, some arrows or other connectors may be used to indicate only the logical flow of the method. For instance, an arrow may indicate a waiting or monitoring period of unspecified duration between enumerated steps of the depicted method. Additionally, the order in which a particular method occurs may or may not strictly adhere to the order of the corresponding steps shown.

Reference throughout this specification to “one example,” “an example,” or similar language means that a particular feature, structure, or characteristic described in connection with the example is included in at least one example. Thus, appearances of the phrases “in one example,” “in an example,” and similar language throughout this specification may, but do not necessarily, all refer to the same example, but mean “one or more but not all examples” unless expressly specified otherwise. The terms “including,” “comprising,” “having,” and variations thereof mean “including but not limited to” unless expressly specified otherwise. An enumerated listing of items does not imply that any or all of the items are mutually exclusive and/or mutually inclusive, unless expressly specified otherwise. The terms “a,” “an,” and “the” also refer to “one or more” unless expressly specified otherwise.

Furthermore, the described features, advantages, and characteristics of the examples may be combined in any suitable manner. One skilled in the relevant art will recognize that the examples may be practiced without one or more of the specific features or advantages of a particular example. In other instances, additional features and advantages may be recognized in certain examples that may not be present in all examples.

These features and advantages of the examples will become more fully apparent from the following description and appended claims, or may be learned by the practice of examples as set forth hereinafter. As will be appreciated by one skilled in the art, aspects of the present invention may be embodied as a system, method, and/or computer program product. Accordingly, aspects of the present invention may take the form of an entirely hardware example, an entirely software example (including firmware, resident software, micro-code, etc.) or an example combining software and hardware aspects that may all generally be referred to herein as a “circuit,” “module,” or “system.” Furthermore, aspects of the present invention may take the form of a computer program product embodied in one or more computer readable medium(s) having program code embodied thereon.

Many of the functional units described in this specification have been labeled as modules, in order to more particularly emphasize their implementation independence. For example, a module may be implemented as a hardware circuit comprising custom very large scale integrated (“VLSI”) circuits or gate arrays, off-the-shelf semiconductors such as logic chips, transistors, or other discrete components. A module may also be implemented in programmable hardware devices such as a field programmable gate array (“FPGA”), programmable array logic, programmable logic devices or the like.

Modules may also be implemented in software for execution by various types of processors. An identified module of program code may, for instance, comprise one or more physical or logical blocks of computer instructions which may, for instance, be organized as an object, procedure, or function. Nevertheless, the executables of an identified module need not be physically located together, but may comprise disparate instructions stored in different locations which, when joined logically together, comprise the module and achieve the stated purpose for the module.

Indeed, a module of program code may be a single instruction, or many instructions, and may even be distributed over several different code segments, among different programs, and across several memory devices. Similarly, operational data may be identified and illustrated herein within modules, and may be embodied in any suitable form and organized within any suitable type of data structure. The operational data may be collected as a single data set, or may be distributed over different locations including over different storage devices, and may exist, at least partially, merely as electronic signals on a system or network. Where a module or portions of a module are implemented in software, the program code may be stored and/or propagated on in one or more computer readable medium(s).

Furthermore, examples may take the form of a program product embodied in one or more computer readable storage devices storing machine readable code, computer readable code, and/or program code, referred hereafter as code. The storage devices, In some examples, are tangible, non-transitory, and/or non-transmission.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (“RAM”), a read-only memory (“ROM”), an erasable programmable read-only memory (“EPROM” or Flash memory), a static random access memory (“SRAM”), a portable compact disc read-only memory (“CD-ROM”), a digital versatile disk (“DVD”), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (“ISA”) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (“LAN”) or a wide area network (“WAN”), or the connection may be made to an external computer (for example, through the Internet using an Internet Service Provider). In some examples, electronic circuitry including, for example, programmable logic circuitry, field-programmable gate arrays (“FPGA”), or programmable logic arrays (“PLA”) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to examples of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The schematic flowchart diagrams and/or schematic block diagrams in the Figures illustrate the architecture, functionality, and operation of possible implementations of apparatuses, systems, methods and computer program products according to various examples of the present invention. In this regard, each block in the schematic flowchart diagrams and/or schematic block diagrams may represent a module, segment, or portion of code, which comprises one or more executable instructions of the program code for implementing the specified logical function(s).

It should also be noted that, in some alternative implementations, the functions noted in the block may occur out of the order noted in the Figures. For example, two blocks shown in succession may, in fact, be executed substantially concurrently, or the blocks may sometimes be executed in the reverse order, depending upon the functionality involved. Other steps and methods may be conceived that are equivalent in function, logic, or effect to one or more blocks, or portions thereof, of the illustrated Figures.

Although various arrow types and line types may be employed in the flowchart and/or block diagrams, they are understood not to limit the scope of the corresponding examples. Indeed, some arrows or other connectors may be used to indicate only the logical flow of the depicted example. For instance, an arrow may indicate a waiting or monitoring period of unspecified duration between enumerated steps of the depicted example. It will also be noted that each block of the block diagrams and/or flowchart diagrams, and combinations of blocks in the block diagrams and/or flowchart diagrams, can be implemented by special purpose hardware-based systems that perform the specified functions or acts, or combinations of special purpose hardware and program code.

The description of elements in each figure may refer to elements of proceeding figures. Like numbers refer to like elements in all figures, including alternate examples of like elements.

As used herein, a list with a conjunction of “and/or” includes any single item in the list or a combination of items in the list. For example, a list of A, B and/or C includes only A, only B, only C, a combination of A and B, a combination of B and C, a combination of A and C or a combination of A, B and C. As used herein, a list using the terminology “one or more of” includes any single item in the list or a combination of items in the list. For example, one or more of A, B and C includes only A, only B, only C, a combination of A and B, a combination of B and C, a combination of A and C or a combination of A, B and C. As used herein, a list using the terminology “one of” includes one and only one of any single item in the list. For example, “one of A, B and C” includes only A, only B or only C and excludes combinations of A, B and C. As used herein, “a member selected from the group consisting of A, B, and C,” includes one and only one of A, B, or C, and excludes combinations of A, B, and C. As used herein, “a member selected from the group consisting of A, B, and C and combinations thereof” includes only A, only B, only C, a combination of A and B, a combination of B and C, a combination of A and C or a combination of A, B and C.

Examples of the present disclosure include a method for calibrating a mercury detection system. The method introducing a gas into a chamber. The gas passes a container within the chamber to form a mixture of mercury from the container and gas. The method includes carrying the mixture of gas and mercury out of the chamber via an input line. The method includes pulling a portion of the mixture out of the input line. The method includes measuring a flow rate of a remaining portion of the mixture in the input line and introducing the remaining portion into an analyzer via an outlet line.

In some examples, the gas is one of a helium carrier gas and a nitrogen carrier gas. In various examples, the method further includes adjusting a rate of mercury flowing out of the container by adjusting a temperature of a permeation oven. In one or more examples, the chamber is housed within the permeation oven. In some examples, measuring the flow rate of the remaining portion of the mixture further includes measuring the flow rate via a venturi flow meter.

In some examples, the method further includes pushing air through an air flow scrubber. In some examples, the air flow scrubber includes activated carbon. In some examples, the method includes, after pushing the air through the air flow scrubber, diluting the mixture of gas and mercury with the air in a mixing container to form a diluted mixture. In some examples, pulling a portion of the mixture out of the input line includes pulling a portion of the diluted mixture out of the input line. In some examples, the method further includes pushing the air through a heated line between the air flow scrubber and the mixing container.

The present disclosure includes a system for calibrating a mercury detection system. The system includes a chamber, a container positioned within the chamber, and an input line connected to the chamber. The system includes a controller configured to introduce a gas into the chamber such that the gas passes the container to form a mixture of mercury from the container and gas. The controller is also configured to push the mixture of gas and mercury out of the chamber via the input line. The system includes a pump configured to pull a portion of the mixture out of the input line. The system includes a flow meter configured to measure a flow rate of a remaining portion of the mixture in the input line. The system includes an outlet line configured to introduce the remaining portion of the mixture into an analyzer.

In some examples, the gas includes one of a helium carrier gas and a nitrogen carrier gas. In some examples, the system further includes a permeation oven within which the chamber is housed. In various examples, the controller is further configured to adjust a rate of mercury flowing out of the container by adjusting a temperature of a permeation oven. In some examples, the flow meter is a venturi flow meter.

In some examples, the pump is a first pump. In various examples, the system further includes an air flow scrubber. In some examples, the air flow scrubber includes activated carbon. In various examples, the system further includes a mixing container fluidically connected to the air flow scrubber and to the input line and a second pump configured to push air through the air flow scrubber and into the mixing container to form a diluted mixture with the mixture of gas and mercury. In some examples, the first pump is configured to pull the portion of the mixture out of the input line by pulling a portion of the diluted mixture out of the input line. In various examples, the system includes a heated line fluidically connected to the mixing container. In some examples, the second pump is configured to push the air out of the air flow scrubber and through the heated line into the mixing container. In some examples, the system includes a heating element a heating element configured to heat the heated line to a temperature of not less than 70 and not greater than 180 degrees Celsius. In some examples, at least one of the first pump and the second pump is configured to adjust a mercury level of the remaining portion by adjusting a flow rate.

In some examples, the analyzer includes a mercury detector configured to detect a concentration of mercury in ambient air, and the system includes a processor configured to calibrate the mercury detector based at least in part on the remaining portion of the mixture. In some examples, the mercury detector is configured to detect a concentration of mercury in ambient air within a given range and the pump is configured to pull the portion of the mixture out of the input line such that a mercury concentration of the remaining portion is not greater than 10 percent outside of the given range.

In some examples, the pump is configured to pull the portion of the mixture out of the input line such that a mercury concentration of the remaining portion of the mixture is not less than 0.1 nanograms per cubic meter. In some examples, the system includes a gas source connected to the chamber. The controller is configured to introduce gas into the chamber from the gas source. In some examples, an inner surface of at least one of the container and the input line comprises at least one of: a fluoropolymer, polytetrafluoroethylene, amorphous silicon dioxide, fused silica, and/or deactivated fused silica. In some examples, the controller is further configured to introduce the gas into the chamber and push the mixture of gas and mercury out of the chamber such that the mixture of gas and mercury flows continuously out of the chamber for a period of not less than 45 minutes.

Mercury can have negative environmental and health effects. Mercury detection systems can help to measure levels of mercury in particular environments and/or substances. Calibrating mercury detection systems helps to improve the accuracy of measurements. Examples of the present disclosure include apparatuses, systems, and methods for calibrating a mercury detection system that help to reduce calibration time and the relevancy of mercury levels produced by diluting an outflow of mercury. Examples of the present disclosure can help to facilitate continuous flow of mercury from a calibration outlet to an analyzer and keep the mercury concentration low enough to help reduce the risk of contaminating the analyzer.

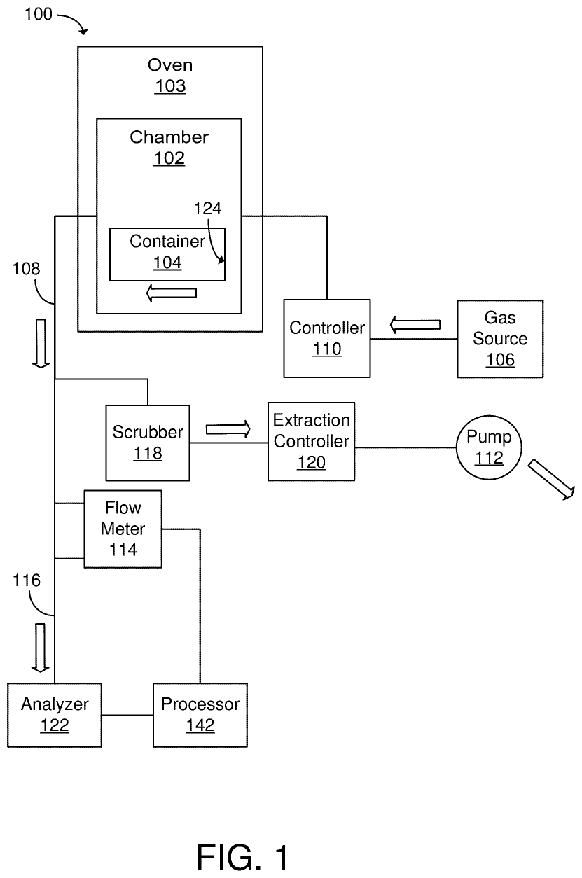

FIG. 1 is a schematic diagram illustrating a system 100 for calibrating a mercury detection system, according to various examples. In some examples, the system 100 includes a chamber 102, a container 104, a gas source 106, a input line 108, a controller 110, a pump 112, a flow meter 114, an outlet line 116, a scrubber 118, an extraction controller 120, an analyzer 122, a processor 142, and/or a combination thereof.

In various examples, mercury is originally contained within the container 104, and various components of the system 100 cause the mercury to flow out of the container 104 and through at least one of: the chamber 102, the input line 108, the scrubber 118, the flow meter 114, the outlet line 116, the analyzer 122, and/or a combination thereof. As used herein, the term “mercury” refers to at least one of elemental mercury or a mercury compound, such as an oxidized mercury compound. In some examples, a mercury compound includes: mercury bromide (HgBr₂), mercury nitrate (Hg(NO₃)₂), mercury chloride (HgCl₂), mercurous chloride (Hg₂Cl₂), mercury oxide (HgO), and/or mercury sulfide (HgS). However, the present disclosure is not limited to such examples. Although the present disclosure describes examples of mercury being in the container 104 and the system 100 causing the mercury to flow, examples of the present disclosure also include methods, systems, and apparatuses for calibrating a detecting system for other types of gases. A person of skill in the art will appreciate that the methods and systems described herein can be applied to calibrate a detector for other types of gases besides mercury.

In some examples, one or more components of the system 100 coming into contact with the mercury include a material that is substantially inert to mercury. Such components include, in some examples, at least one of: the chamber 102, inner surfaces of the chamber 102, a coating of the inner walls of the chamber 102, the container 104, inner walls 124 of the container 104, the input line 108, inner surfaces of the input line 108, the outlet line 116, and/or any combination thereof. The inert material, in some examples, includes at least one of: polytetrafluoroethylene (e.g., Teflon®), perfluoroalkoxy, a fluoropolymer, a polymer, carbon, fluorine, silicon, silica, a metallic material coated with silica, silica-coated stainless steel, Sulfinert®, amorphous silicon dioxide, fused silica, deactivated fused silica, and/or any combination thereof. In some examples, the substantially inert material is less likely to react with mercury compounds, helping to prevent sticking of the mercury compounds to components of the system 100, such as inner walls of the chamber 102, inner walls 124 of the container, and/or the lines 108, 116.

In some examples, the chamber 102 is housed within a permeation oven 103. In some or more examples, the container 104 is tubular. In some examples, the chamber 102 includes a tube, and the container 104 is another tube within the larger tube. In some examples, the chamber 102 is made of a same or similar material to the input line 108 and/or outlet line 116. In some examples, the container 104 is positioned and/or housed within the chamber 102. In some examples, walls of the container 104 are permeable by mercury compounds. In some examples, the container 104 is made of a polymeric material. In some examples, the container 104 is made of a porous material, allowing mercury compounds to escape from the container 104. In some examples, the container 104 includes a plug located at each end of the container 104 such that the mercury flows radially outward from the inside of the container 104.

In some examples, the gas source 106 is in fluid communication with the chamber 102 and supplies gas to the chamber 102, as controlled by the controller 110. In some examples, the gas is a carrier gas. In some examples, the gas includes at least one of: helium, nitrogen, and/or a combination thereof. In some examples, the gas includes one of a helium carrier gas and a nitrogen carrier gas. In some examples, the controller 110 is configured to introduce gas from the gas source 106 into the chamber 102 such that the gas passes the container 104 to form a mixture of gas and mercury. In one or more examples, the container 104 is at least partially permeable to the mixture of gas and mercury. In some examples, the mercury flows through the walls of the container 104 and pervades the chamber 102 such that the mercury and gas mix in the chamber 102. In some examples, the mixture is then passed into the input line 108 and through other components of the system 100. In some examples, the gas flows around the outside of the container 104, drawing mercury compounds out of the container 104. In various examples, the gas flows externally to the container 104 but within the chamber 102 along a length of the container 104, creating a mixture of the gas and mercury. In other examples, the gas flows directly into and through the container 104.

In some examples, the system 100 includes one or more components configured to measure a mass of content within the container 104. In various examples, such components are communicably coupled to one or more other components of the system 100, such as the processor 142 and/or the controller 110. In some examples, the controller 110 adjusts the flow rate of the mercury based at least in part on the original mass of content within the container 104. In various examples, the controller 110 is configured to terminate a flow of gas into the chamber 102 in response to determining that the mass of contents in the container 104 has fallen below a threshold value.

In one or more examples, the rate of the mercury flowing from the container 104 and into the chamber 102 is adjustable by adjusting a temperature within the container 104. In some examples, the chamber 102 includes and/or is exposed to one or more energy emitters, such as heat sources of the permeation oven 103. In some examples, the energy emitters are configured to adjust the temperature within the chamber 102 by adjusting the temperature of the permeation oven 103 while the chamber 102 is housed within the permeation oven 103. In some examples, the system 100 includes a controller configured to adjust the temperature within the chamber 102. In various examples, the walls of the chamber 102 are not permeable to the mixture, thus helping to prevent the mercury from contaminating the permeation oven 103.

In some examples, the controller is configured to control energy emitters within the chamber 102 to increase temperature within the chamber 102 and thus increase a rate of mercury components traveling from within the container 104, out of the container 104, and into the surrounding chamber 102. In some examples, the heating elements are configured to heat the container 104 to increase the porosity of the container 104, thus increasing the rate of mercury flowing out of the container 104. In some examples, the heating elements heat the container 104 to a temperature of not less than 50 degrees Celsius (“° C.”). In some examples, the heating elements are configured to heat the container 104 to a temperature of not less than 40° C. and not greater than 80° C. In some examples, the heating element includes at least one of: a convection oven, a fan, a thermoelectric heater, a temperature controller, and/or a combination thereof.

In some examples, the input line 108 is connected to the chamber 102. In some examples, the input line 108 is also connected to the container 104. In one or more examples, the controller 110 is configured to carry (e.g., by pushing) the mixture of gas and mercury out of the chamber 102 via the input line 108. In some examples, the controller 110 is configured to adjust a mercury content of the gas-mercury mixture by adjusting a flow rate of the gas from the gas source 106, thus adjusting a dilution level of the gas in the gas-mercury mixture. In some examples, at least one of the pump 112 and the air flow pump 236 is controllable by the controller 110 to adjust the mercury level of the remaining portion by adjusting the flow rate.

In some examples, the controller 110 is configured to cause the mixture of gas and mercury to flow from the container 104 to the outlet line 116 continuously over a gas flow period. As such, examples of the present disclosure can help to provide a continuous flow of mercury rather than a pulsed flow. In various examples, that gas flow period is a period of time selected to help provide equilibrium between the mercury compounds and the inner walls of the chamber 102 and inner surfaces of the outlet line 116 and input line 108. In one or more examples, the gas flow period is not less than 30 minutes. In one or more examples, the gas flow period is not less than 1 hour. In some examples, the gas flow period is not less than 2 hours. In some examples, the gas flow period is not less than 45 minutes. In one or more examples, the gas flow period is not less than 30 minutes and not greater than 3 hours. In some examples, the gas flow period is not less than 1 hour and not greater than 24 hours.

In some examples, the pump 112 is configured to pull a portion of the mixture out of the input line 108. In one or more examples, the pump 112 is configured to pull the portion of the mixture out of the input line 108 such that a remaining portion of the mixture remains in the input line 108. In some examples, the system 100 also includes a scrubber 118 and/or an extraction controller 120. In some examples, the extraction controller 120 is configured to control the flow of the mixture extracted from the input line 108. In some examples, the scrubber 118 is configured to remove an impurity (e.g., mercury, reactive gases, ozone, and/or a combination thereof) from the mixture before the pump 112 pulls the extracted portion of the mixture to a vent and/or exhaust component.

In some examples, the remaining portion of the mixture is less than the extracted portion of the mixture by total mass. In some examples, the extraction controller 120 is configured to extract not less than 50% of the gas-mercury mixture flow through the input line 108. In some examples, the extraction controller 120 is configured to extracted not less than 80% of the gas-mercury mixture. In some examples, the extraction controller 120 is configured to extract not less than 90% of the mixture flow. In some examples, the extraction controller 120 is configured to extract not less than 95% of the mixture flow. In some examples, the extraction controller 120 is configured to extract not less than 99% of the mixture flow. In some examples, the extraction controller 120 is configured to extract not less than 90% and not greater than 99.5% of the mixture flow.

In some examples, the extraction controller 120 is configured to extract a portion of the mixture flow such that the total mercury within the remaining mixture is within a range. In some examples, that range encompasses the range of mercury measurements that the mercury detection system being calibrated by the system 100 may make. In some examples, the range is not less than 0.01 nanograms (“ng”) and not greater than 5 ng of mercury per cubic meter of air. In some examples, the range is not less than 0.1 nanograms and not greater than 5 ng of mercury per cubic meter of air. In some examples, the extraction controller 120 is configured to extract a portion of the mixture such that the remaining portion of the mixture includes at least 1 ng of mercury per cubic meter. In various examples, the range is not less than 5 ng and not greater than 10 ng per cubic meter. In some examples, the given range is not less than 2 ng and not greater than 10 ng per cubic meter. In some examples, the given range is not less than 0.2 ng and not greater than 0.5 ng per cubic meter.

In some examples, the extraction controller 120 is configured to measure a flow rate of the remaining portion in the input line 108. In some examples, the extraction controller 120 determines the flow rate and/or mercury content of the remaining portion by subtracting a flow rate of the extracted portion moving through the scrubber 118 from an extracted portion that moves through a vent connected to the pump 112.

In some examples, the remaining portion flows toward the flow meter 114, which is located downstream of the extraction. In some examples, the flow meter 114 is configured to measure a flow rate of a remaining portion of the mixture in the input line 108. In some examples, the flow meter 114 is a Venturi flow meter. In some examples, the flow meter 114 includes a tube with a constricted section, or throat. In some examples, the flow meter 114 includes one or more pressure sensors configured to measure differences in pressure between an inlet of the flow meter 114 and the throat. In some examples, the flow meter 114 is configured to measure and/or calculate the flow rate based at least in part on at least one of: the difference, a density of the remaining portion, a cross-sectional area of the throat, a diameter of the tube, and/or a combination thereof. In some examples, the tube is made of and/or coated with a material that is substantially inert to mercury, as described above. In some examples, the tube is made of stainless steel.

In some examples, the remaining portion passes the flow meter 114 and flows into the outlet line 116. In various examples, the outlet line 116 is fluidically connected to the input line 108. In various examples, the outlet line 116 and input line 108 are portions of the same line. In some examples, the outlet line 116 is configured to introduce the remaining portion into an analyzer 122 after the flow meter 114 has measured the flow rate. In some examples, one or more of the controller 110 and the extraction controller 120 are configured to adjust an amount of mercury compounds outputted to an analyzer 122 by adjusting flow rates. In some examples, the outlet line 116 is a heated outlet line. In one or more examples, the outlet line 116 is removably coupled to an inlet of the analyzer 122.

In some examples, the controller 110 is configured to stop the gas flow in response to determining that the mercury concentration in the remaining portion is stable. In various examples, the system 100 determines that the mercury concentration is stable by continuously monitoring the flow meter 114 measurements over the gas flow period. In some examples, in response to the mercury concentration level in the remaining portion of the mixture varying by less than a given percentage over a monitoring interval, the controller 110 stops the gas flow. In some examples, the monitoring period is not less than 30 minutes and not greater than 3 hours. In various examples, the percentage is not less than 1 percent and not greater than 10 percent.

In some examples, one or more of the controller 110 and the extraction controller 120 includes a mass flow controller, a proportional control valve, a sensor measuring air flow, a controller, a physical valve, and/or a combination thereof. In some examples, the controller 110 includes a control valve configured to constrict the flow of the gas from the gas source 106 to regulate the volume of gas flowing into the chamber 102. In some examples, the extraction controller 120 controls the flow rate and/or pressure of the mixture being removed from the input line 108 via communication with the pump 112. In some examples, the extraction controller 120 is configured to send electrical signals to the pump 112 to control the speed and/or power output of the pump 112.

In some examples, the analyzer 122 is an analyzer of a mercury detection system. In some examples, the analyzer 122 is a mercury analyzer, such as a continuous emission monitoring system or an ambient mercury vapor analyzer. In one or more examples, the analyzer 122 includes one or more of: a sensor, a plasmonic sensor, a dual beam ultraviolet (UV) absorption sensor, a cold vapor atomic fluorescence spectroscopy sensor, a mass spectrometer, a heating element, a mercury collector, or a combination thereof. In various examples, the analyzer 122 is configured to detect a concentration of mercury in a sample of ambient air. In some examples, the analyzer 122 detects the concentration level by collecting mercury from ambient air in a collector, heating the mercury collector to a given temperature, capturing mercury compounds released from the collector via a sample trap, heating the sample trap such that the mercury compounds flow into a mass spectrometer, and measuring the mercury compounds via the mass spectrometer.

In some examples, the analyzer 122 is configured to detect a mercury concentration for ambient air having a mercury concentration within a given range. In one or more examples, the extraction controller 120 is configured to adjust the concentration of mercury outputted to the analyzer 122 such that the concentration of mercury in the fluid outputted to the analyzer 122 is within the given range.

In various examples, the analyzer 122 is located downstream of the flow meter 114. In some examples, the analyzer 122 is communicably coupled to the flow meter 114. In some examples, the analyzer 122 is configured to determine the mercury concentration based at least in part on a flow measurement from the flow meter 114. In some examples, the analyzer 122 is configured to determine the mass flow rate of mercury based at least in part on a product of the mercury concentration and the flow rate from the flow meter 114. In some examples, the system 100 is configured to calibrate the analyzer 122 based at least in part on measurements from the flow meter 114.

In some examples, the system 100 includes a processor 142 configured to calibrate the mercury detector of the analyzer 122 based at least in part on a mercury level in the remaining portion of the mixture. In some examples, the processor 142 is part of the analyzer 122. In one or more examples, the processor 142 is external to the analyzer 122. In various examples, the analyzer 122 measures a mercury level in the remaining portion of the mixture. In one or more examples, the processor 142 is configured to perform the calibration by the mercury level detected by the analyzer 122 to the mercury level measured by the flow meter 114.

In some examples, the processor 142 is configured to determine whether a difference between the these two measurements is above a threshold. In one or more examples, if the difference is above the threshold, the processor 142 calculates a correction factor and/or adjusts the analyzer 122's internal parameters, such as the sensor's gain or offset, to bring its reading into agreement with the value measured by the flow meter 114. Thus, the system 100 can help to improve the analyzer 122's probably of providing accurate, traceable measurements of mercury concentration in the ambient air.

In some examples, the analyzer 122 includes a display and/or is communicably connected with a display. In various examples, the analyzer 122 outputs at least one of the following through the display: a measured mercury level in ambient air, a measured mercury level in the calibration sample (the remaining portion of the mercury mixture fed into the analyzer 122 by the system 100), a temperature of the calibration sample, or a combination thereof. In some examples, the display is part of a user interface (UI). In various examples, the system 100 receives input from a user via the UI. In various examples, the analyzer 122 is in communication with at least one of: the controller 110, the extraction controller 120, the permeation oven 103, or a combination thereof. In some examples, the controller 110 and/or the extraction controller 120 are configured to adjust a mercury level of the calibration sample based at least in part on input received through the UI.

In one or more examples, at least one of the controller 110 and the extraction controller 120 receives, through the UI, a given range of mercury levels which the analyzer 122 is to detect. In some examples, one or more of the controller 110 and the extraction controller 120 is configured to adjust the levels of mercury in the calibration sample to be within the given range. In one or more examples, the permeation oven 103 is configured to adjust the calibration sample to a temperature selected by a user through the UI. In some examples, the analyzer 122 is in communication with the flow meter 114 and is configured to display measurements of the mercury levels in the calibration sample through the UI. In some examples, the analyzer 122 is configured to display its own measurements of mercury levels in ambient air through the UI.

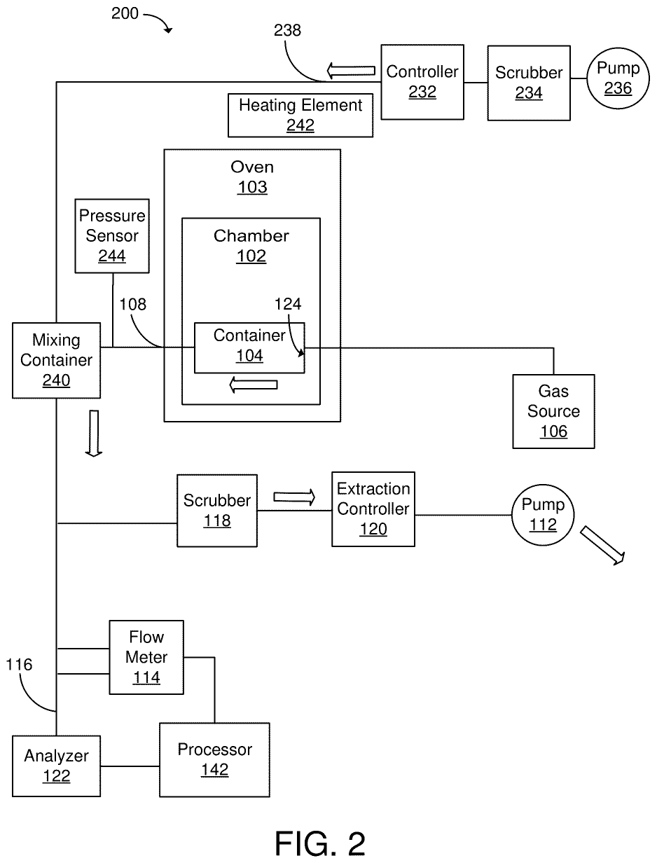

FIG. 2 is a schematic diagram illustrating a system 200 for calibrating a mercury detection system using additional air flow, according to various examples. The system 200 is a variation of the system 100. In some examples, the system 200 includes components of the system 100, such as the chamber 102, the container 104, the gas source 106, the input line 108, the controller 110, the pump 112, the flow meter 114, and the outlet line 116, which are substantially similar to those described above in relation to the system 100 of FIG. 1. In some examples, the system 200 includes additional components, such as an air flow controller 232, an air flow scrubber 234, an air flow pump 236, a heated line 238, a mixing container 240, a pressure sensor 244, and/or a combination thereof.

In some examples, the air flow pump 236 is configured to move additional air into and through the air flow scrubber 234. In some examples, the air flow scrubber 234 is configured to remove an impurity or contaminant in the air, such as mercury, reactive gases, ozone, or a combination thereof, from the additional air. In some examples, the air flow scrubber 234 includes a scrubbing material, a first filter, and/or a second filter. In one or more examples, the first filter and the second filter are positioned on opposing ends of the air flow scrubber 234. In some examples, the filters hold the scrubbing material in place. In various examples, the scrubbing material is activated carbon. In some examples, the air flow scrubber 234 including activated carbon as a scrubbing material is referred to as a carbon scrubber.

In some examples, the air flow controller 232 is configured to move the additional air into and/or through the heated line 238 after the additional air has been at least partially purified by the air flow scrubber 234. In various examples, the air flow controller 232 is configured to control the air flow pump 236 to move the additional air into and through the heated line 238. In some examples, the air flow controller 232 includes a mass flow controller, a proportional control valve, a sensor measuring air flow, a controller, a physical valve, and/or a combination thereof. In some examples, the air flow controller 232 includes a control valve configured to constrict the flow of the additional air to regulate the volume of additional air pumped into the heated line 238. In some examples, the air flow controller 232 controls the flow rate and/or pressure of the additional air being pumped into the heated line 238 via communication with the air flow pump 236. In some examples, the air flow controller 232 is configured to send electrical signals to the air flow pump 236 to control the speed and/or power output of the air flow pump 236.

In some examples, the system 200 includes a pressure sensor 244 connected to the input line 108. In various examples, the pressure sensor 244 is configured to measure a flow of gas out of the chamber 102 and/or permeation oven 103. In one or more examples, the pressure sensor 244 is in communication with at least one of the controller 232 and the extraction controller 120. In some examples, the controller 232 and/or the extraction controller 120 are configured to control flow rates based at least in part on measurements received from the pressure sensor 244.

In one or more examples, the heated line 238 is configured to heat the additional air to a threshold temperature before the air comes into contact with mercury compounds from the gas-mercury mixture formed in the chamber 102. In some examples, the threshold temperature is approximately 150° C. In some examples, the threshold temperature is not less than 150° C. In one or more examples, the threshold temperature is not less than 100 and not greater than 200° C. In one or more examples, the threshold temperature is not less than 120 and not greater than 180° C. In one or more examples, the threshold temperature is not less than 70 and not greater than 180° C. In various examples, the heated line 238 extends between the air flow scrubber 234 and a mixing container 240, which is also referred to herein as a mixing chamber or a mixing tube. In some examples, the heated line 238 is configured to carry the air from the air flow scrubber 234 and into the mixing container 240. In one or more examples, a length of the heated line 238, from an end receiving the air to a juncture between the input line 108 and the heated line 238, is selected to help facilitate heating of the air to the threshold temperature. In some examples, the length is not less than 0.6 and not greater than 2.6 meters.

In some examples, the system 200 includes one or more heating elements 242 configured to heat the air within the heated line 238. Such heating elements 242 include, in some examples, one or more of: resistance heaters wrapped around the heated line 238, heat emitters, steam injectors, a heated fluid supply, and/or a combination thereof.

In some examples, the mixture of gas and mercury formed in the chamber 102 meets the scrubbed air. In some examples, the input line 108 carrying the mixture joins the heated line 238. In other examples, the input line 108 carrying the mixture and the heated line 238 are both fluidically connected to the mixing container 240. In some examples, the mixing container 240 combines the scrubbed air with the gas-mercury mixture. In various examples, after the air flow pump 236 pushes the air through the air flow scrubber 234, the air flow pump 236 dilutes the mixture of gas and mercury with the scrubbed air in the mixing container 240 to form a diluted mixture. In some examples, the mixing container 240 helps to more uniformly dilute the gas-mercury mixture with the scrubbed air after the air is pushed through the air flow scrubber 234. Referring to FIG. 2, in some examples, the mixing container 240 is a tube in fluid communication with an outlet of the chamber 102 and to an inlet that receives the scrubbed air.

In some examples, the air flow controller 232 creates a diluted mixture in the mixing container 240. In some examples, the extraction controller 120 and pump 112 are configured to extract a portion of that diluted mixture out of the input line 108, while a remaining portion of the diluted mixture flows to the flow meter 114, as described in connection with FIG. 1.

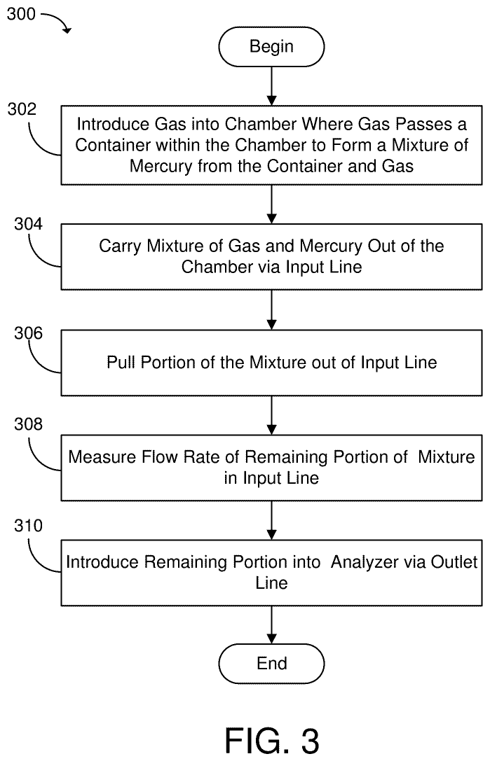

FIG. 3 is a schematic flow chart diagram illustrating a method 300 for calibrating a mercury detection system, according to various examples. In some examples, one or more steps of the method 300 are performed by components of the system 100 and/or the system 200. In some examples, the method 300 begins and includes introducing 302 a gas into a chamber 102. The gas passes a container 104 within the chamber to form a mixture of mercury from the container 104 and gas. The method 300 includes carrying 304 the mixture of gas and mercury out of the chamber 102 via an input line 108. The method 300 includes pulling 306 a portion of the mixture out of the input line 108. The method 300 includes measuring 308 a flow rate of a remaining portion of the mixture in the input line 108 and introducing 310 the remaining portion into an analyzer 122 via an outlet line 116, and the method 300 ends.

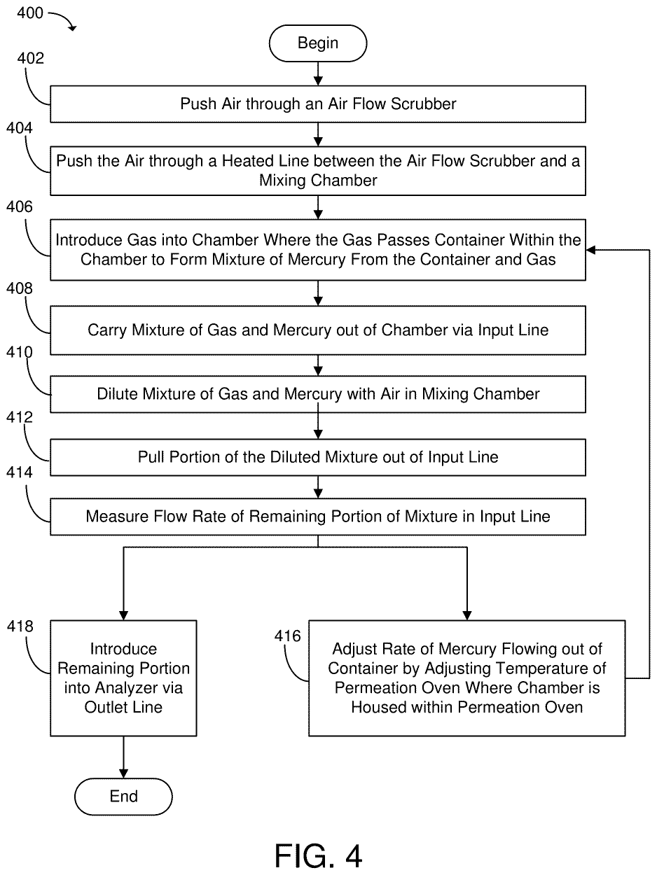

FIG. 4 is a schematic flow chart diagram illustrating one example of a method 400 for calibrating a mercury detection system with additional airflow. In some examples, one or more steps of the method 400 are performed by components of the system 100 and/or the system 200. In various examples, the method 400 begins and includes pushing 402 air through an air flow scrubber 234. In some examples, the method 400 includes pushing 404 the air through a heated line 238 between the air flow scrubber 234 and a mixing container 240. In various examples, the method 400 includes introducing 406 gas into a chamber 102. The gas passes a container 104 within the chamber 102 to form a mixture of mercury from the container 104 and gas. The method 400 includes carrying 408 the mixture of gas and mercury out of the chamber 102 via an input line 108.

In some examples, the method 400 includes diluting 410 the mixture of gas and mercury with air in a mixing container 240. In some examples, the method 400 includes pulling 412 a portion of the diluted mixture of out of the input line 108. In various examples, the method 400 includes measuring 414 the flow rate of the remaining portion of the mixture in the input line 108. In some examples, the method 400 includes adjusting 416 the rate of mercury flowing out of the container 104 by adjusting the temperature of a permeation oven 103. The chamber 102 is housed within the permeation oven 103. In some examples, the method 400 includes to step 408. In some examples, after step 414, the method 400 includes introducing 418 the remaining portion of the mixture into an analyzer 122 via an outlet line 116, and the method 400 ends.

The present invention may be embodied in other specific forms without departing from its spirit or essential characteristics. The described examples are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. A method for calibrating a mercury detection system, the method comprising:
introducing a gas into a chamber, wherein the gas passes a container within the chamber to form a mixture of mercury from the container and gas;
carrying the mixture of gas and mercury out of the chamber via an input line;
pulling a portion of the mixture out of the input line;
measuring a flow rate of a remaining portion of the mixture in the input line; and
introducing the remaining portion into an analyzer via an outlet line.

2. The method of claim 1, wherein the gas comprises one of a helium carrier gas and a nitrogen carrier gas.

3. The method of claim 1, further comprising adjusting a rate of mercury flowing out of the container by adjusting a temperature of a permeation oven, wherein the chamber is housed within the permeation oven.

4. The method of claim 1, wherein measuring the flow rate of the remaining portion of the mixture further comprises measuring the flow rate via a venturi flow meter.

5. The method of claim 1, further comprising:
pushing air through an air flow scrubber, the air flow scrubber comprising activated carbon; and
after pushing the air through the air flow scrubber, diluting the mixture of gas and mercury with the air in a mixing container to form a diluted mixture, wherein pulling a portion of the mixture out of the input line comprises pulling a portion of the diluted mixture out of the input line.

6. The method of claim 5, further comprising pushing the air through a heated line between the air flow scrubber and the mixing container.

7. A system for calibrating a mercury detection system, comprising:
a chamber;
a container positioned within the chamber;
an input line connected to the chamber;
a controller configured to:
introduce a gas into the chamber such that the gas passes the container to form a mixture of mercury from the container and gas; and
push the mixture of gas and mercury out of the chamber via the input line;
a pump configured to pull a portion of the mixture out of the input line;
a flow meter configured to measure a flow rate of a remaining portion of the mixture in the input line; and
an outlet line configured to introduce the remaining portion of the mixture into an analyzer.

8. The system of claim 7, wherein the gas comprises one of a helium carrier gas and a nitrogen carrier gas.

9. The system of claim 7, further comprising a permeation oven within which the chamber is housed, wherein the controller is further configured to adjust a rate of mercury flowing out of the container by adjusting a temperature of a permeation oven.

10. The system of claim 7, wherein the flow meter comprises a venturi flow meter.

11. The system of claim 7, wherein:
the pump comprises a first pump;
the system further comprises:
an air flow scrubber comprising activated carbon;
a mixing container fluidically connected to the air flow scrubber and to the input line; and
a second pump configured to push air through the air flow scrubber and into the mixing container to form a diluted mixture with the mixture of gas and mercury; and
the first pump is configured to pull the portion of the mixture out of the input line by pulling a portion of the diluted mixture out of the input line.

12. The system of claim 11, further comprising a heated line fluidically connected to the mixing container, wherein the second pump is configured to push the air out of the air flow scrubber and through the heated line into the mixing container.

13. The system of claim 12, further comprising a heating element configured to heat the heated line to a temperature of not less than 70 and not greater than 180 degrees Celsius.

14. The system of claim 11, wherein at least one of the first pump and the second pump is configured to adjust a mercury level of the remaining portion by adjusting a flow rate.

15. The system of claim 7, wherein the analyzer comprises a mercury detector configured to detect a concentration of mercury in ambient air and the system further comprises a processor configured to calibrate the mercury detector based at least in part on the remaining portion of the mixture.

16. The system of claim 15, wherein the mercury detector is configured to detect a concentration of mercury in ambient air within a given range and the pump is configured to pull the portion of the mixture out of the input line such that a mercury concentration of the remaining portion is not greater than 10 percent outside of the given range.

17. The system of claim 7, wherein the pump is configured to pull the portion of the mixture out of the input line such that a mercury concentration of the remaining portion of the mixture is not less than 0.1 nanograms per cubic meter.

18. The system of claim 7, further comprising a gas source connected to the chamber, wherein the controller is configured to introduce the gas into the chamber from the gas source.

19. The system of claim 7, wherein an inner surface of at least one of the container and the input line comprises at least one of: a fluoropolymer, polytetrafluoroethylene, amorphous silicon dioxide, fused silica, and/or deactivated fused silica.

20. The system of claim 7, wherein the controller is further configured to introduce the gas into the chamber and push the mixture of gas and mercury out of the chamber such that the mixture of gas and mercury flows continuously out of the chamber for a period of not less than 45 minutes.
